(19) United States
(12) Patent Application Publication
(10) Pub. No.: US 2023/0290482 A1
(43) Pub. Date: Sep. 14, 2023

(54) Digital Apparatus and Application for Treating Social Communication Disorder

(71) Applicant: S-Alpha Therapeutics, Inc., Seoul (KR)

(72) Inventors: Seung Eun Choi, Seoul (KR); Myoung Joon Kim, Seoul (KR)

(73) Assignee: S-Alpha Therapeutics, Inc., Seoul (KR)

(21) Appl. No.: 18/019,617

(22) PCT Filed: Aug. 4, 2021
(86) PCT No.: PCT/KR2021/010257

§ 371 Date: Feb. 3, 2023

Related U.S. Application Data
(60) Provisional application No. 63/061,092, filed on Aug. 4, 2020.

(51) Int. Cl. G16H 20/70; G16H 50/20; G16H 40/67; G06F 3/16

(52) U.S. Cl. CPC G16H 20/70; G16H 50/20; G16H 40/67; G06F 3/16

(57) ABSTRACT

Systems and methods for treating social communication disorder are provided. A system may include a digital apparatus, which may include a digital instruction generation unit configured to generate instructions in real-time or near-real-time for the user to follow to treat social communication disorder based on a mechanism of action (MOA) in and a therapeutic hypothesis for the social communication disorder, and an outcome collection unit configured to collect the user's execution outcomes of the digital instructions. The system may also include a healthcare provider portal for a healthcare provider to manage their patients and/or an administrative portal for an administrator to manage healthcare providers.

### TECHNICAL FIELD

The present disclosure relates to digital therapeutics (hereinafter referred to as DTx) intended for social communication disorder therapy, which includes inhibition of progression of social communication disorder. The present disclosure also relates to systems that integrate digital therapeutics with one or both of a healthcare provider portal and an administrative portal to treat social communication disorder in a patient. In particular, embodiments of the present disclosure may comprise deducing a mechanism of action (hereinafter referred to as MOA) in a subject having social communication disorder through a literature search and expert reviews of basic scientific articles and related clinical trial articles to find the mechanism of action in social communication disorder, and establishing a therapeutic hypothesis and a digital therapeutic hypothesis for inhibiting progression of social communication disorder in a subject and treating the social communication disorder based on these findings. The present disclosure also relates to a rational design of a digital application for clinically verifying a digital therapeutic hypothesis for social communication disorder in a subject and realizing the digital therapeutic hypothesis for digital therapeutics. The present disclosure also relates to a digital apparatus and an application for inhibiting progression of social communication disorder in a subject and treating the social communication disorder based on this rational design.

### BACKGROUND ART

Social communication disorder (SCD) broadly describes a disruption of the normal physical or mental processes associated with social interaction (e.g., speech style and context, rules for linguistic politeness), social cognition (e.g., emotional competence, understanding emotions of self and others), and pragmatics (e.g., communicative intentions, body language, eye contact). A social communication disorder may be a distinct diagnosis or may occur within the context of other conditions, such as autism spectrum disorder (ASD), specific language impairment (SLI), learning disabilities (LD), language learning disabilities (LLD), intellectual disabilities (ID), developmental disabilities (DD), attention deficit hyperactivity disorder (ADHD), and traumatic brain injury (TBI). For example, with respect to ASD, social communication disorders are a defining feature. Although the incidence and prevalence of SCD can be difficult to determine (e.g., due to clinical studies drawing on varied populations and being conducted using varying criteria for making a clinical diagnosis of SCD), as many as 1 in 3 children may have some form of SCD. However, there is no highly reliable therapeutic method that subjects who have been diagnosed with SCD can use to inhibit progression of and treat SCD.

In some instances, SCD is caused by a failure of the pragmatic-semantic process (e.g., a partially or completely diminished coordination between verbal and non-verbal responses), leading the affected individual to have a lack of confidence, depression, and the like. DTx can help by restoring coordination between verbal and non-verbal responses. However, there are very few DTx in this field, and these programs are unable of receiving input from the subject without his or her active use of an input device (such as a mouse, keyboard or touch screen etc.). As such, these programs are limited to those subjects who are capable of using an input device. Furthermore, current methods of diagnosing, inhibiting, and/or treating of social communication disorders are not based on real-time or near-real-time events. For example, diagnosing an individual with SCD or determining a treatment plan may be based on controlled social interaction between the subject and a professional, rather than being based on real-life events.

Accordingly, there exists a need for DTx that are capable of (i) receiving input from the subject (or another individual involved in social communication with the subject) without the need for his or her active use of an input device (e.g., based on sound or gestures), and (ii) providing instructions based on the input to the subject in real-time or near-real-time to treat SCD.

### DISCLOSURE OF INVENTION

### Brief Description of Drawings

The above and other objects, features and advantages of the present disclosure will become more apparent to those of ordinary skill in the art by describing in detail exemplary embodiments thereof with reference to the attached drawings, in which:

FIG. 1 illustrates a comparison of the exemplary symptoms, and target treatment for healthy individuals and individuals having Autism, ADD/ADHD, or SCD;

FIG. 2 illustrates an square diagram predicting the exemplary situations in which a subject having SCD may have an un healthy social interaction (e.g., exhibit sadness, or anger) based (i) on the type of environment (e.g., formal or informal Environment), and (ii) the type of communication (e.g., predictable or unpredictable communication);

FIG. 3 illustrates the exemplary pragmatic-semantic process, and how one or both of (i) continuous and supplementary behavioral information, and (ii) ACTH- or Enkephalinase-related action language can be used to treat SCD in a subject.

FIG. 4 illustrates an exemplary decision tree for how a subject having SCD may respond during an event, and how a digital application of the present disclosure can aid the subject in responding appropriately during the event;

FIG. 5 illustrates an exemplary diagram of how a digital application of the present disclosure uses one or more of pre-event, real-time or near-real-time event, and post-event information to process data and generate instructions to maximize a subject's response in real-time to treat SCD is the subject;

FIG. 6 illustrates an exemplary diagram of how a digital application of the present disclosure uses real-time or near-real-time event information to process data and generate instructions to maximize a subject's response in real-time to treat SCD;

FIG. 7 illustrates an exemplary diagram for scoring based on a sum of evaluated values for different group 1 parameters analyzed from inputted voice.

FIG. 8 illustrates an exemplary scoring method based on a sum of evaluated values for group 1 parameters (e.g., anger, sadness, tension, pleasant and excitation parameters in the inputted voice compared to a standard voice in response to the event).

FIG. 9 illustrates exemplary group 1 parameters for inputted voice, group 2 parameters for contents in a conversation, and group 3 parameters for tone in a conversation. Scoring may be based on a total sum of each sum of evaluated values for a different group.

FIG. 10 is a diagram showing an exemplary feedback loop for a digital apparatus and an digital application for treating social communication disorder according to one embodiment of the present disclosure;

FIG. 11 is a flowchart illustrating exemplary operations in a digital application for treating social communication disorder according to one embodiment of the present disclosure;

FIG. 12 is a diagram showing an exemplary hardware configuration of the digital apparatus for treating social communication disorder according to one embodiment of the present disclosure;

FIG. 13 is a table showing exemplary privileges for the doctors using the healthcare provider portal and the administrators using the administrative portal.

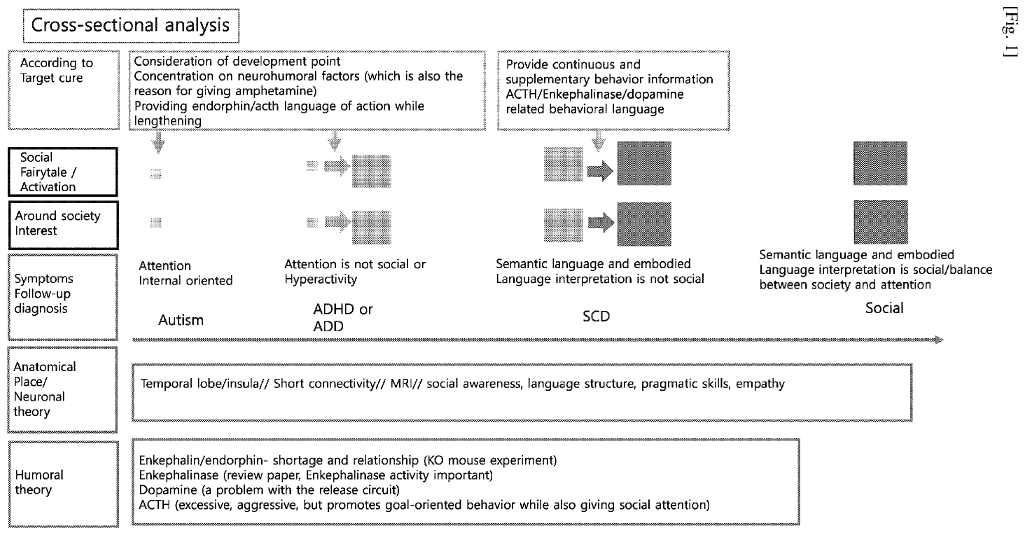

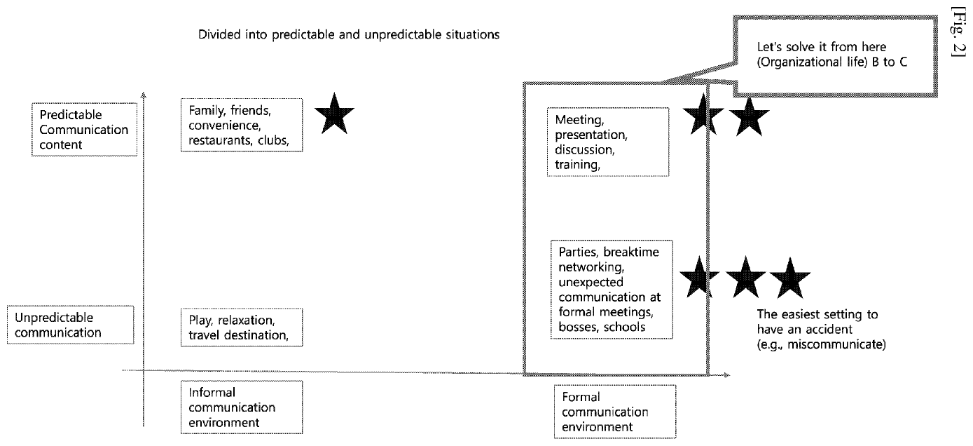

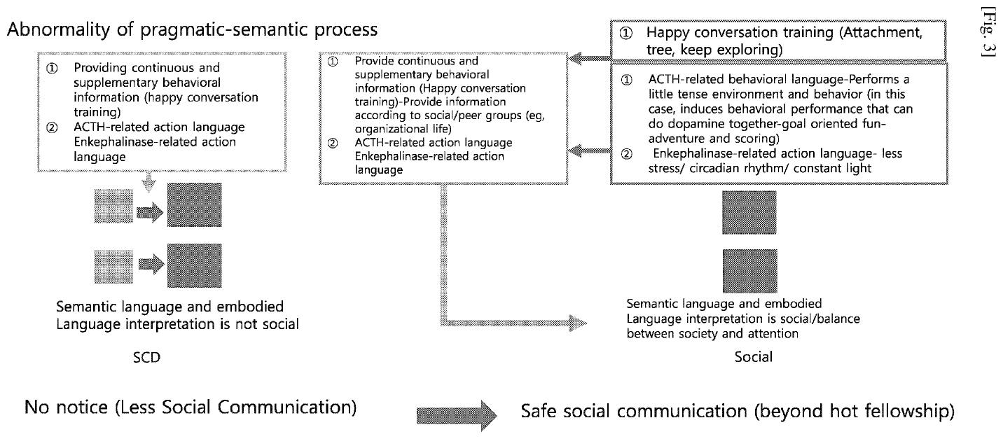

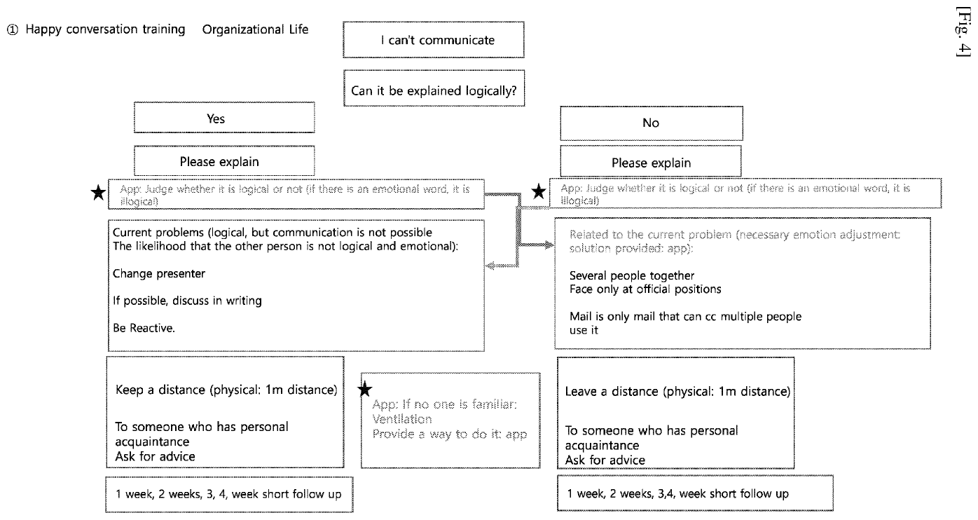

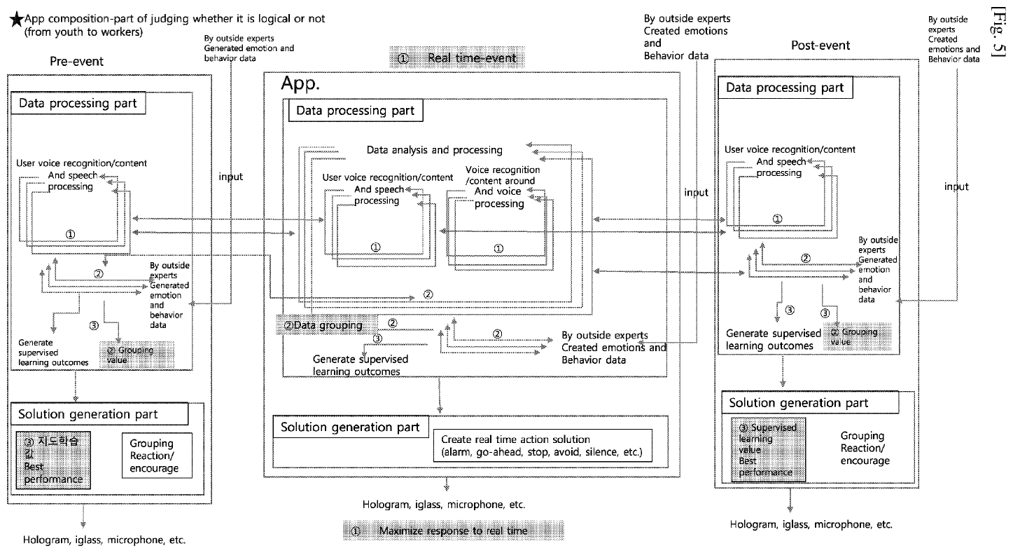

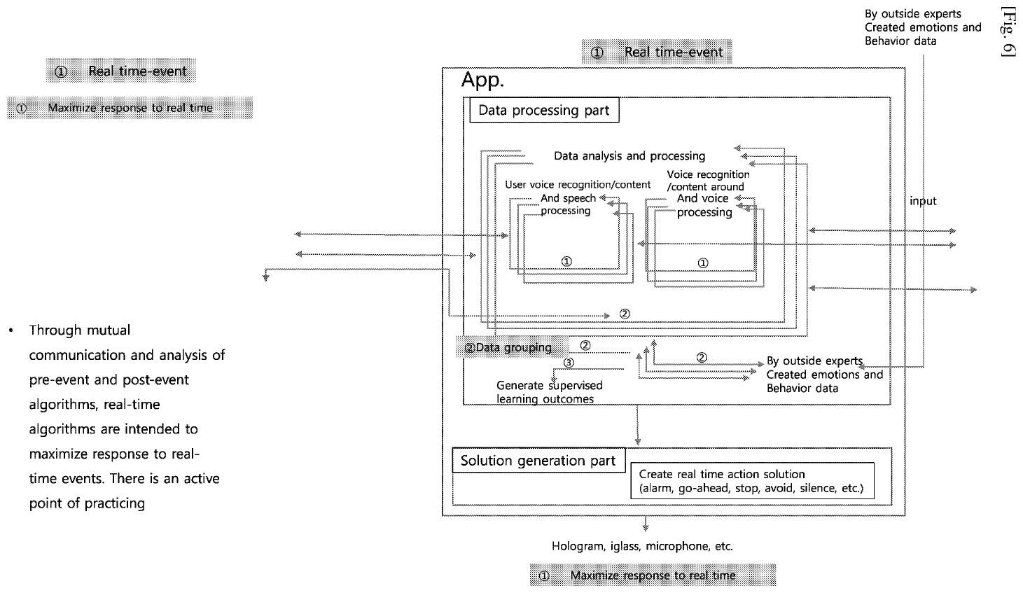

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### MODE FOR THE INVENTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may mean a range of ±20%, ±10%, or ±5% of a given numeric value.

As used herein, the term “real-time” or “near-real-time” generally refer to the characteristic of occurring contemporaneously with an event. For example, in certain embodiments of the present disclosure, one or more instructions can be provided to a subject in real-time. As used herein, the term “real-time” can refer to a characteristic of being simultaneous with an event, or within 1 second of an event, within 5 seconds of an event, within 10 seconds of an event, within 15 seconds of an event, within 30 seconds of an event, within 1 minute of an event, within 2 minutes of an event, or within 5 minutes of an event.

Overview

With reference to the appended drawings, exemplary embodiments of the present disclosure will be described in detail below. To aid in understanding the present disclosure, like numbers refer to like elements throughout the description of the figures, and the description of the same elements will be not reiterated.

In certain aspects, the present disclosure provides a method of treating social communication disorder (SCD) in a subject in need thereof. In certain embodiments, the method comprises detecting, with an electronic device, sound or gesture of social communication with the subject in an event, wherein the electronic device comprises a sensor for sensing the sound or gesture of the social communication with the subject in the event. In certain embodiments, the method comprises providing one or more first instructions for the subject to improve social interaction, social cognition, and/or pragmatics based on one or more characteristics of the sound or gesture of the social communication. Generally, the one or more instructions can be independently selected from the group consisting of an alarm, a silent alarm or a vibration, an instruction to proceed, an instruction to stop, and instruction to avoid, and an instruction to maintain silence.

A patient or subject treated by any of the methods, systems, or digital applications described herein may be of any age and may be an adult or child, however the methods and systems of the present disclosure are particularly suitable for students over the age of 5 and adults over the age of 21. In some cases, the patient or subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 5 and 65 years old, between 20 and 65 years old, or between 30 and 65 years old). In some embodiments, the patient or subject is a child. In some embodiments, the patient or subject is a child, and is supervised by an adult when using the methods, systems, or digital applications of the present disclosure.

In certain embodiments, the method comprises detecting, with an electronic device, sound of social communication with the subject in an event. It will be understood that an electronic device can generally refer to any device capable of detecting sound or gestures involved in social communication. Non-limiting examples of an electronic device include a smartphone (e.g., an Apple iPhone™), a smartwatch (e.g., an Apple Watch™), a tablet (e.g., an Apple iPad™), a laptop computer (e.g., an Apple MacBook™), a smart eyeglass (e.g., Apple Glass™), and the like. In certain embodiments an electronic device can comprise a plurality of electronic devices (e.g., a primary electronic device and a secondary electronic device). A person of skill in the art will appreciate that any number of devices may be used, and that those device may be wirelessly linked to transmit and receive information (e.g., between the devices, or from a device to a server). It is contemplated that different devices may be used in various embodiments of the present disclosure in order to take advantage of the unique features of each device. A subject can carry a smart phone as a primary electronic device and a smart watch as a secondary electronic device. For example, while a smartphone may be used to analyze sound of social communication and determine, based on the sound, one or more instructions for the subject to follow, a smartwatch may be used to detect the sound of social communication since the smartwatch is a wearable technology disposed on the surface of the body closer to the source of the sound (e.g., and not in a pocket where sound may be more difficult to detect). In another example, while a smartphone may be used to analyze gestures of social communication and determine, based on the gestures, one or more instructions for the subject to follow, a smart eyeglass may be used to detect the sound of social communication since the smart eyeglass is a wearable technology disposed on the surface of the body and positioned to readily observe gestures in the social communication (e.g., and not in a pocket where the gestures may be more difficult to detect).

In certain embodiments, the electronic device comprises a sensor for sensing sound of the social communication with the subject in the event. In certain embodiments, the electronic device comprises a sensor for sensing gestures of the social communication with the subject in the event. Non-limiting examples of sensors include a camera, a photo cell, a microphone, an activity sensor, a motion sensor, a sound meter, an acoustic sensor, an optical sensor, an ambient light sensor, an infrared sensor, an environmental sensor, a temperature sensor, a thermometer, a pressure sensor, and an accelerometer. In certain embodiments, an electronic device comprises a single sensor. In certain embodiments, an electronic device comprises 2 sensors, 3 sensors, 4 sensors, 5 sensors, 6 sensors, 7 sensors, 8 sensors, 9 sensors, 10 sensors, or more than 10 sensors. For example, an electronic device can comprise 2 sensors (e.g., a camera and a microphone).

In certain embodiments, the electronic device comprises a sensor for sensing sound of the social communication with the subject in the event. Sound of social communication can refer, for example, to a human voice. In certain embodiments, the human voice is that of the subject. In other embodiments, the human voice is that of an individual involved in social communication with the subject. In certain embodiments, the sound is an ambient sound (e.g., voices of nearby individuals who are not involved in social communication with the subject). For example, in certain embodiments, a sensor can be configured to detect ambient sounds in order to reduce the ambient sounds or enhance the sounds associated with the social communication between the subject and another individual. In certain embodiments, an electronic device comprises a sensor for sensing sound of social communication, and then the sound is analyzed determine one or more characteristics of the sound. Non-limiting examples of the characteristics of a sound of social communication can include one or more of vocabulary, syntax, phonology, voice wavering, voice frequency, rate of speech, word spacing, tone, voice pitch, voice amplitude, and coherence. Voice can be used to judge anger, irritability, and mood through amplitude, transliteration, and the like. Facial expressions can be used to judge a pleasant expression, an annoying expression, and the like. It will be understood that any method available in the art can be used to analyze sound of social communication to determine a characteristic of the sound. For example, US Publication No. 20190385066, which is incorporated by reference herein in its entirety, relates to artificial intelligence technology, a robot, and a method for predicting an emotional state by the robot. In another example, US Publication No. 20180174020, which is incorporated by reference herein in its entirety, relates to systems and methods for emotionally intelligent automatic chat. The system and method provide an emotionally intelligent automatic (or artificial intelligence) chat by knowing the context and emotion of the conversation with the user. Based on these decisions, the system and method can select one or more responses from a response database to responses to user queries. In addition, the systems and methods can be modified or trained based on user feedback or environmental feedback. In yet another example, U.S. Publication No. 20180181854, which is incorporated by reference herein in its entirety, relates to a system and method using artificial emotional intelligence to receive a variety of input data, process the input data, return a computational response stimulus and analyze the input data. Various electronic devices may be used to obtain input data regarding a specific user, multiple users, or environments. This input data, which can consist of voice tones, facial expressions, social media profiles, and surrounding environment data, can be compared to historical data related to a particular user, user group, or environment. The systems and methods of this document can employ artificial intelligence to evaluate the collected data and provide stimuli to users or groups of users. Response stimulation can be in the form of music, quotations, pictures, jokes, suggestions, etc. In yet another example, U.S. Publication No. 20190286996, which is incorporated by reference herein in its entirety, relates to a human machine interactive method based on artificial intelligence and a human machine interactive device based on artificial intelligence.

Similarly, in certain embodiments, the electronic device comprises a sensor for sensing gestures of the social communication with the subject in the event. Gestures of social communication can refer, for example, to eye contact by the subject or an individual involved in social communication with the subject, eye movement by the subject or an individual involved in social communication with the subject, facial expressions by the subject or an individual involved in social communication with the subject, body language by the subject or an individual involved in social communication with the subject, and hand gestures by the subject or an individual involved in social communication with the subject.

In certain embodiments, the sound or the gesture of the social communication is categorized. In certain embodiments, the sound or the gesture of the social communication is categorized as being associated with one or more of a standard response, a sarcastic response, a cynical response, an angry response, a sad response, a tense response, a pleasant response, an excited response, an accurate response, or an appropriate response. For example, the sound or gesture of the social communication can be categorized as a standard response if the sound or gesture is routinely performed by the subject in course of their daily life. Categorization of the sound or the gesture can be performed, for example, using outside experts to categorize whether a particular sound or gesture is associated with a given type of response (e.g., a standard response, a sarcastic response, a cynical response, an angry response, a sad response, a tense response, a pleasant response, an excited response, an accurate response, or an appropriate response). In another example, categorization of the sound or the gesture can be performed using a reviewer to categorize whether a particular sound or gesture is associated with a given type of response (e.g., a standard response, a sarcastic response, a cynical response, an angry response, a sad response, a tense response, a pleasant response, an excited response, an accurate response, or an appropriate response). In another example, categorization of the sound or the gesture can be performed using a healthcare provider to categorize whether a particular sound or gesture is associated with a given type of response (e.g., a standard response, a sarcastic response, a cynical response, an angry response, a sad response, a tense response, a pleasant response, an excited response, an accurate response, or an appropriate response). In another example, categorization of the sound or the gesture can be performed using behavioral data to categorize whether a particular sound or gesture is associated with a given type of response (e.g., a standard response, a sarcastic response, a cynical response, an angry response, a sad response, a tense response, a pleasant response, an excited response, an accurate response, or an appropriate response). In another example, categorization of the sound or the gesture can be performed using a machine learning model trained to use behavioral data to categorize whether a particular sound or gesture is associated with a given type of response (e.g., a standard response, a sarcastic response, a cynical response, an angry response, a sad response, a tense response, a pleasant response, an excited response, an accurate response, or an appropriate response). In another example, categorization of the sound or the gesture can be performed using an artificial intelligence to categorize whether a particular sound or gesture is associated with a given type of response (e.g., a standard response, a sarcastic response, a cynical response, an angry response, a sad response, a tense response, a pleasant response, an excited response, an accurate response, or an appropriate response). In yet another example, categorization of the sound or the gesture can be performed using data obtained from the subject following the event or the pre-event to categorize whether a particular sound or gesture is associated with a given type of response (e.g., a standard response, a sarcastic response, a cynical response, an angry response, a sad response, a tense response, a pleasant response, an excited response, an accurate response, or an appropriate response). For example, following an event, the subject can input data to the digital application characterizing a sound or gesture of the social communication as being associated with one or more of a standard response, a sarcastic response, a cynical response, an angry response, a sad response, a tense response, a pleasant response, an excited response, an accurate response, and an appropriate response. Without limitation, a particular sound or gesture can be categorized two or more of a standard response, a sarcastic response, a cynical response, an angry response, a sad response, a tense response, a pleasant response, an excited response, an accurate response, and an appropriate response. [37] In certain embodiments, the method comprises providing one or more first instructions (e.g., based on the categorization) for the subject to improve social interaction, social cognition, and/or pragmatics based on one or more characteristics of the sound of the social communication. The instructions can be provided to the subject in real-time, or near-real-time, of an event. As used herein, the term “real-time” or “near-real-time” generally refer to the characteristic of occurring contemporaneously with an event. For example, in certain embodiments of the present disclosure, one or more instructions can be provided to a subject in real-time. “Real-time” can refer to a characteristic of being simultaneous with an event, or within 1 second of an event, within 5 seconds of an event, within 10 seconds of an event, within 15 seconds of an event, within 30 seconds of an event, within 1 minute of an event, within 2 minutes of an event, or within 5 minutes of an event. “Real-time” can also refer to a characteristic of being simultaneous with an pre-event, or within 1 second of an pre-event, within 5 seconds of an pre-event, within 10 seconds of an pre-event, within 15 seconds of an pre-event, within 30 seconds of an pre-event, within 1 minute of an pre-event, within 2 minutes of an pre-event, or within 5 minutes of an pre-event. An event can generally refer to an imaginary scenario (e.g., a fabricated event, a pre-event, or a practice event that a subject is exposed to using the electronic device), or a real-world event.

In certain embodiments, the one or more first instructions for the subject to improve social interaction, social cognition, and/or pragmatics are determined based on the categorization of the sound or gestures of the social communication.

In certain embodiments, the electronic device comprises a digital instruction generation unit configured to generate one or more instructions for treating SCD based on a mechanism of action (MOA) in and a therapeutic hypothesis for the SCD, and provide the one or more instructions to the subject. In some embodiments, the digital apparats comprises an outcome collection unit configured to collect the subject's execution outcomes of the digital instructions. In some embodiments, a digital application of the present disclosure can provide one or more instructions to the subject (e.g., to walk around, or to think positively) to increase dopamine levels in the subject in order to improve confidence in the subject. In some embodiments, a digital application of the present disclosure can provide one or more instructions to the subject (e.g., to perform aerobic exercise) to increase oxytocin levels in the subject in order to increase sociality. In some embodiments, a digital application of the present disclosure can provide one or more other instructions to the subject, for example, conducting collaborative tasks, training to improve language recognition, training to understand metaphors and/or jokes, training to manage aggressive emotions, or training to predict or foresee an attack (verbal or physical) from another individual. In certain embodiments, a digital application of the present disclosure can provide one or more instructions to the subject to regulate (e.g., increase, decrease, or maintain) one or more of GABA levels, glutamate levels, serotonin levels, dopamine levels, acetylcholine levels, oxytocin levels, arginine-vasopressin levels, melatonin levels, neuropeptide beta-endorphin levels, pentapeptide metenkephalin levels, encephalin levels, and adrenocorticotropin hormone levels in the body of the subject.

In certain embodiments, the social communication by the subject can be scored by comparing the one or more characteristics with a reference standard. In certain embodiments, the reference standard is determined using a pre-trained machine learning model. In certain embodiments, the reference standard is determined using a pre-trained machine learning model that is trained using a training data set comprising at least one of responses by administrators, healthy individuals and/or responses by individuals having the SCD.

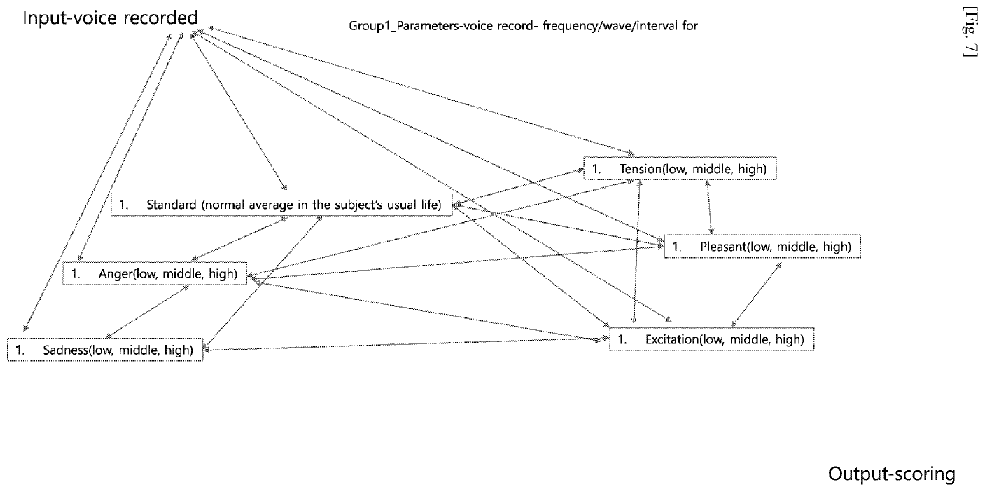

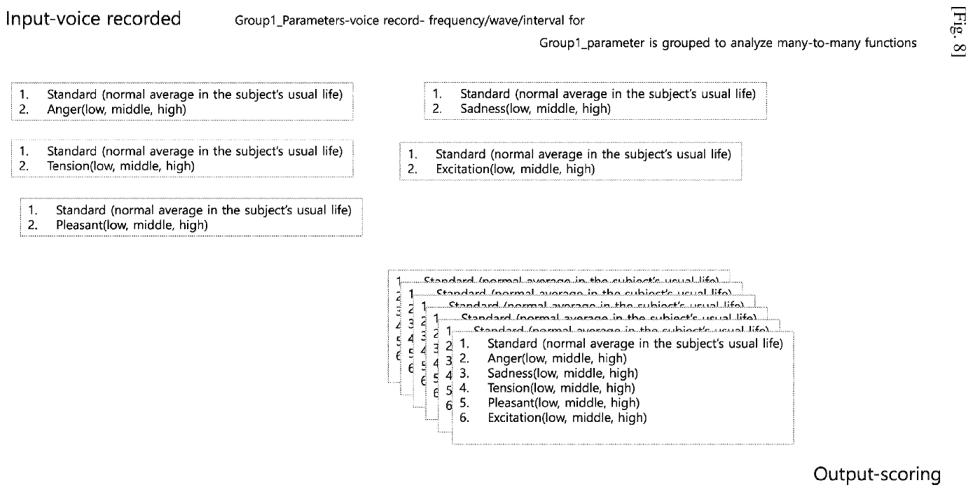

FIGS. 7-9 illustrate an exemplary scoring process in which inputted voice, contents of a conversation and tone of a subject in the conversation are analyzed based on different grouped parameters. Predetermined scores are assigned to predetermined ranges for parameters. For example, when a voice is inputted, the “Anger” parameter is set to be “low” when the volume of the inputted voice is from 1 to 300. One output scoring figure may be produced within a group of parameters, and one total output scoring figure may be produced for all groups.

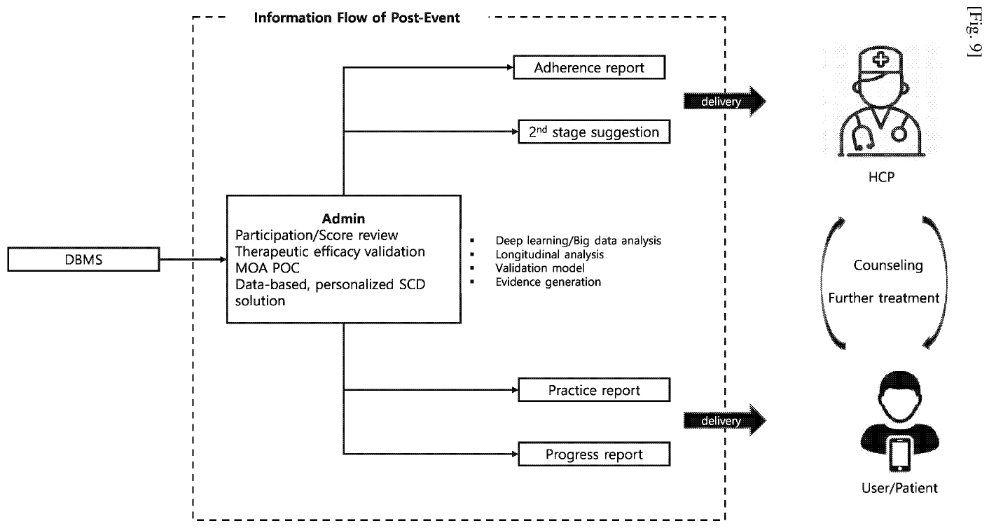

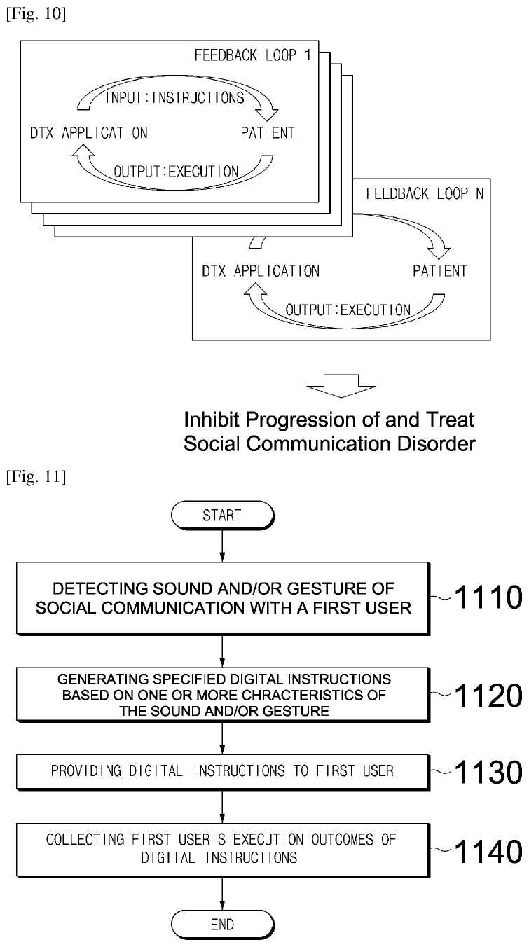

FIG. 10 is a diagram showing a feedback loop for the electronic device and the application for treating social communication disorder according to one embodiment of the present disclosure. Referring to FIG. 10, the inhibition of the progression of and the treatment of social communication disorder are shown to be achieved by repeatedly executing a single feedback loop several times to regulate the biochemical factors.

Inhibitory and therapeutic effects on progression of the social communication disorder may be more effectively achieved by gradual improvement of an instruction-execution cycle in the feedback loop, compared to the simply repeated instruction-execution cycle during the corresponding course of therapy. For example, the digital instructions and the execution outcomes for the first cycle are given as input values and output values in a single loop, but new digital instructions may be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the electronic device and the application for treating social communication disorder according to one embodiment of the present disclosure, the patient's digital instructions provided in the previous cycle (for example, a N−1^{st} cycle), and the data on instruction execution outcomes may be used to calculate the patient's digital instructions and execution outcomes in this cycle (for example, a N^{th} cycle). That is, the digital instructions in the next loop may be generated based on the patient's digital instructions and execution outcomes of the digital instructions calculated in the previous loop. In this case, various algorithms and statistical models may be used for the feedback process, when necessary. As described above, in the electronic device and the application for treating social communication disorder according to one embodiment of the present disclosure, it is possible to optimize the patient-customized digital instructions suitable for the patient through the rapid feedback loop.

FIG. 11 is a flowchart illustrating operations in the digital application for treating social communication disorder according to one embodiment of the present disclosure. Referring to FIG. 11, the digital application for treating social communication disorder according to one embodiment of the present disclosure may first detect sound and/or gesture of social communication with a first user (1110).

Next, in 1120, specified digital instructions may be generated based on the one or more instructions. 1120 may generate a one or more instructions by applying imaginary parameters about the patient's environments, behaviors, emotions, and cognition to the mechanism of action in and the therapeutic hypothesis for social communication disorder. In this case, in 1120, the one or more instructions may be generated based on the biochemical factors (for example, GABA, glutamate, serotonin, dopamine, acetylcholine, oxytocin, arginine-vasopressin, melatonin, neuropeptide beta-endorphin, pentapeptide metenkephalin, encephalin, or adrenocorticotropin hormone) for social communication disorder. Meanwhile, in 1120, the one or more instructions may be generated based on the inputs from the healthcare provider or expert reviewer. In this case, a one or more instructions may be generated based on the information collected by the doctor when diagnosing a patient, and the prescription outcomes recorded based on the information. Also, in 1120, the one or more instructions may be generated based on the information (for example, basal factors, medical information, digital therapeutics literacy, etc.) received from the patient.

Then, the digital instructions may be provided to a patient (1130). In this case, the digital instructions may be provided in the form of digital instructions which are associated with behaviors and in which the patient's instruction adherence may be monitored using a sensor, or provided in the form of digital instructions in which a patient is allowed to directly input the execution outcomes. Generally, the one or more instructions can be independently selected from the group consisting of an alarm, a silent alarm or a vibration, an instruction to proceed, an instruction to stop, and instruction to avoid, and an instruction to maintain silence.

After the patient executes the presented digital instructions, the patient's execution outcomes of the digital instructions may be collected (1140). In 1140, the execution outcomes of the digital instructions may be collected by monitoring the patient's adherence to the digital instructions as described above, or allowing the patient to input the execution outcomes of the digital instructions.

Meanwhile, the digital application for treating social communication disorder according to one embodiment of the present disclosure may repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions provided in the previous cycle.

As described above, according to the digital application for treating social communication disorder according to one embodiment of the present disclosure, the reliability of the inhibition of progression of and treatment of social communication disorder may be ensured by deducing the mechanism of action in and the therapeutic hypothesis for social communication disorder in consideration of the biochemical factors for social communication disorder, presenting the digital instructions to a patient based on the mechanism of action in and the therapeutic hypothesis for social communication disorder, and collecting and analyzing the outcomes of the digital instructions.

Although the electronic device and the application for treating social communication disorder according to one embodiment of the present disclosure have been described in terms of social communication disorder therapy, the present disclosure is not limited thereto. For the other diseases other than the social communication disorder, the digital therapy may be executed substantially in the same manner as described above.

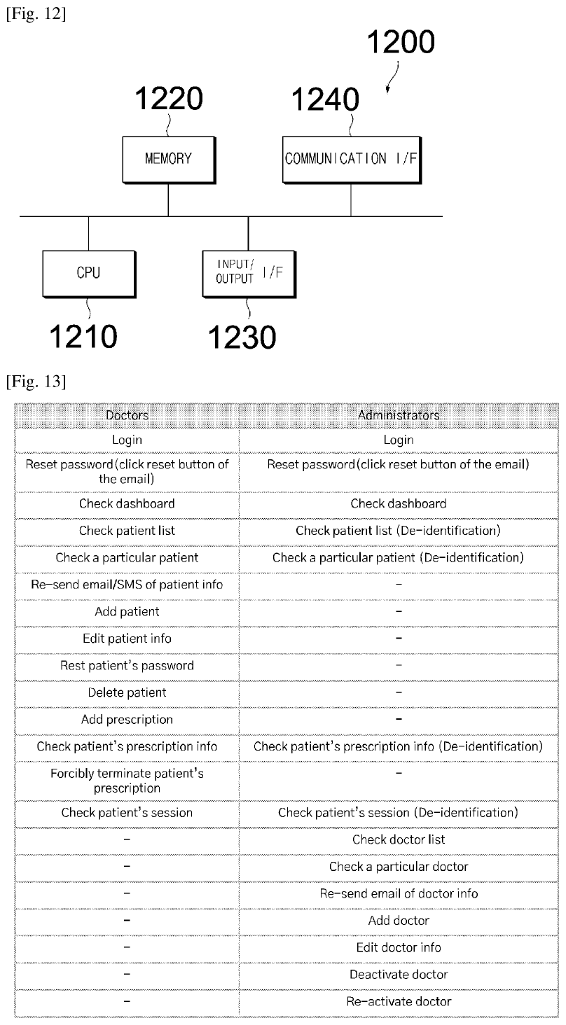

FIG. 12 is a diagram showing a hardware configuration of the electronic device for treating social communication disorder according to one embodiment of the present disclosure.

Referring to FIG. 12, hardware 1200 of the electronic device for treating social communication disorder according to one embodiment of the present disclosure may include a CPU 1210, a memory 1220, an input/output I/F 1230, and a communication I/F 1240.

The CPU 1210 may be a processor configured to execute a digital application for treating social communication disorder stored in the memory 1220, process various data for treating digital social communication disorder and execute functions associated with the digital social communication disorder therapy. That is, the CPU 1210 may act to execute functions by executing the digital application for treating social communication disorder stored in the memory 1220.

The memory 1220 may have a digital application for treating social communication disorder stored therein. Also, the memory 1220 may include the data used for the digital social communication disorder therapy included in the database, for example, the patient's digital instructions and instruction execution outcomes, the patient's medical information, and the like.

A plurality of such memories 1220 may be provided, when necessary. The memory 1220 may be a volatile memory or a non-volatile memory. When the memory 1220 is a volatile memory, RAM, DRAM, SRAM, and the like may be used as the memory 1220. When the memory 1220 is a non-volatile memory, ROM, PROM, EAROM, EPROM, EEPROM, a flash memory, and the like may be used as the memory 1220. Examples of the memories 1220 as listed above are given by way of illustration only, and are not intended to limit the present disclosure.

The input/output I/F 1230 may provide an interface in which input apparatuses (not shown) such as a keyboard, a mouse, a touch panel, and the like, and output apparatuses such as a display (not shown), and the like may transmit and receive data (e.g., wirelessly or by hardline) to the CPU 1210.

The communication I/F 1240 is configured to transmit and receive various types of data to/from a server, and may be one of various apparatuses capable of supporting wire or wireless communication. For example, the types of data on the aforementioned digital behavior-based therapy may be received from a separately available external server through the communication I/F 1240.

According to the electronic device and the application for treating, ameliorating, or preventing social communication disorder according to the present disclosure, a reliable electronic device and application capable of inhibiting progression of and treating social communication disorder may be provided by deducing a mechanism of action in social communication disorder and a therapeutic hypothesis and a digital therapeutic hypothesis for social communication disorder in consideration of biochemical factors for progression of social communication disorder, presenting digital instructions to a patient, and collecting and analyzing execution outcomes of the digital instructions.

In some aspects, the present disclosure provides a system for treating social communication disorder (SCD) in a subject in need thereof. In some embodiments, the system comprises an electronic device. In some embodiments, the electronic device is configured to detect sound of social communication with the subject in an event, wherein the electronic device comprises a sensor for sensing the sound of the social communication with the subject in the event. In some embodiments, the electronic device is configured to provide one or more first instructions for the subject to improve social interaction, social cognition, and/or pragmatics based on one or more characteristics of the sound of the social communication. In some embodiments, the system comprises a healthcare provider portal configured to provide one or more options to a healthcare provider to perform one or more tasks to prescribe treatment for social communication disorder (SCD) in the subject based on information received from the electronic device. In some embodiments, the system comprises an administrative portal configured to provide one or more options to an administrator of the system to perform one or more tasks to manage access to the system by the healthcare provider.

In some embodiments, the present disclosure provides a system for treating social communication disorder, the system comprising an administrative portal (e.g., Administrator's web), a healthcare provider portal (e.g., Doctor's web) and a digital apparatus configured to execute a digital application (e.g., an application or ‘app’) for treating social communication disorder in a subject. Among other things, the Administrator's portal allows an administrator to issue doctor accounts, review doctor information, and review de-identified patient information. Among other things, the Healthcare Provider's portal allows a healthcare provider (e.g., a doctor) to issue patient accounts, and review patient information (e.g., age, prescription information, and status for having completed one or more pre-event social communication practice sessions). Among other things, the digital application allows a patient access to complete one or more pre-event social communication practice sessions.

In some embodiments, the present disclosure provides an execution flow for login verification during a splash process at the starting of the digital application. Similarly, the present disclosure provides an execution flow for prescription verification during a splash process at the starting of the digital application. The prescription verification process may comprise, for example, determining if the treatment period has expired, or determining if, based on the prescription, the subject's sessions for the day have been completed (e.g., the subject is compliant with the prescription). In such instances, the digital apparatus may notify the subject that there are no pre-event social communication practice sessions available to be completed.

In some embodiments, the healthcare provider portal provides a healthcare provider with one or more options, and the one or more options provided to the healthcare provider are selected from the group consisting of adding or removing the subject, viewing or editing personal information for the subject, viewing adherence information for the subject, viewing a result of the subject for one or more at least partially completed pre-event social communication practice sessions, prescribing one or more pre-event social communication practice sessions to the subject, altering a prescription for one or more pre-event social communication practice sessions, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed pre-event social communication practice sessions to be performed by the subject, and a number of scheduled or prescribed pre-event social communication practice sessions to be performed by the subject per day. In some embodiments, the one or more options comprise the viewing the adherence information, and the adherence information of the subject comprises one or more of a number of scheduled or prescribed pre-event social communication practice sessions completed by the subject, and a calendar identifying one or more days on which the subject completed, partially completed, or did not complete one or more scheduled or prescribed pre-event social communication practice sessions. In some embodiments, the one or more options comprise the viewing the result of the subject, and the result of the subject for one or more at least partially completed pre-event social communication practice sessions comprises one or more selected from the group consisting of a time at which the subject started a scheduled or prescribed pre-event social communication practice session, a time at which the subject ended a scheduled or prescribed pre-event social communication practice session, and an indicator of whether the scheduled or prescribed pre-event social communication practice session was fully or partially completed.

In some embodiments, the present disclosure provides a dashboard of a healthcare provider portal. (1) The number of all patients associated with the present doctor's account. A graph may be used to show the number of patients who have opened the digital application for patient per day in the most recent 90 days. The number of patients in progress may also be viewed. A graph may be used to show the number of patients who have completed the sessions per day in the most recent 90 days. In some embodiments, the present disclosure provides a patient tab in a healthcare provider portal, the patient tab displaying a list of patients. For example, the present disclosure provides (1) Patient ID (the unique identification number temporarily given to each patient when adding them on the list), (2) Patient Name, (3) Search bar for searching by ID, Name, Email, Memo, etc., and (4) Add New Patient button for adding new patients. In some embodiments, the present disclosure provides a patient tab in a healthcare provider portal, the patient tab displaying detailed information on a given patient. For example, the present disclosure provides (1) detailed patient information, (2) a button for editing patient information, (3) prescription information, (4) a button for adding a new prescription, (5) a progress status for different each prescription, and (6) a button or link for sending an email to the patient. In some embodiments, the present disclosure provides a patient tab in a healthcare provider portal for adding a new patient. For example, the present disclosure provides (1) a button for adding a new patient, and (3) an error message is displayed when required patient information has not been provided. In some embodiments, the present disclosure provides a patient tab in a healthcare provider portal for editing information of an existing patient. For example, the present disclosure provides (1) a button or link for resetting a password, (2) a button for deleting a given patient, and (3) a button for saving changes. In some embodiments, the present disclosure provides a patient tab in a healthcare provider portal that displays detailed prescription information for a given patient. For example, the present disclosure provides (1) a button for editing prescription information, (2) the duration of the sessions attended by the patient or subject, and (3) an overview the treatment progress. Seven days are represented as a line or row of 7 squares. For 12 weeks, each 6 weeks may be presented separately. Different colors may be used to discern session statuses (e.g., grey for sessions not started, red for sessions not attended, yellow for sessions partially attended, and green for sessions fully attended). In some embodiments, the present disclosure provides a patient tab in a healthcare provider portal for editing prescription information for a given patient.

In some embodiments, the administrative portal provides an administrator with one or more options, and the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed pre-event social communication practice sessions, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed pre-event social communication practice sessions completed by the subject, and a calendar identifying one or more days on which the subject completed, partially completed, or did not complete one or more scheduled or prescribed pre-event social communication practice sessions. In some embodiments, the one or more options comprise the viewing the result of the subject, and the result of the subject for one or more at least partially completed pre-event social communication practice sessions comprises one or more selected from the group consisting of a time at which the subject started a scheduled or prescribed pre-event social communication practice session, a time at which the subject ended a scheduled or prescribed pre-event social communication practice session, and an indicator of whether the scheduled or prescribed pre-event social communication practice session was fully or partially completed.

In some embodiments, the present disclosure provides (A) a dashboard of an administrative portal. For example, the present disclosure provides (1) the number of doctors. A graph may be used to show the number of doctors that have visited the digital application per day in the most recent 90 days, (2) The number of all patients associated with the any doctor's account. A graph may be used to show the number of patients who have opened the digital application for patient per day in the most recent 90 days. The number of patients in progress may also be viewed. A graph may be used to show the number of patients who have completed the sessions per day in the most recent 90 days. In some embodiments, the present disclosure provides a doctor tab in an administrative portal, the doctor tab displaying a list of doctors. For example, the present disclosure provides (1) a search bar for searching for various doctors by name, email, etc., (2) a button for adding a new doctor, (3) the doctor's ID, (4) a button for viewing detailed doctor information, and (5) deactivated doctor accounts. In some embodiments, the present disclosure provides a doctor tab in an administrative portal, the doctor tab displaying a list of patients being cared for by a given doctor, with patient-identifying information redacted (*). For example, the present disclosure provides (1) the doctor's account information, (2) a button for editing the doctor's account information, (3) a list of patients being cared for by the doctor, (4) a list of patient ID numbers, (5) a link or button for sending the doctor a registration email, (6) a notification that the doctor's account has been deactivated, which only appears for deactivated accounts, and (7 and 8) redacted or de-identified patient information. In some embodiments, the present disclosure provides a doctor tab in an administrative portal for adding a new doctor. In some embodiments, the present disclosure provides a doctor tab in an administrative portal for editing information of an existing doctor, including activating or deactivating a doctor's account. In some embodiments, the present disclosure provides a patient tab in an administrative portal that displays information for one or more patients, wherein sensitive information is redacted. In some embodiments, the present disclosure provides a patient tab in an administrative portal that displays detailed patient or prescription information for a given patient. In some embodiments, the present disclosure provides a patient tab in an administrative portal that displays detailed prescription information for a given patient. FIG. 13 provides a table showing privileges for the doctors using the healthcare provider portal and the administrators using the administrative portal.

In some aspects, the present disclosure provides a computing system for treating social communication disorder (SCD) in a subject in need thereof. In some embodiments, the computing system comprises a sensor for detecting sound of social communication with the subject in an event. In some embodiments, the computing system comprises a digital instruction generation unit configured to provide, to the subject, one or more first instructions for the subject to follow to improve social interaction, social cognition, and/or pragmatics, said one or more instructions based on one or more characteristics of the sound of the social communication.

Any of the computer systems mentioned herein can utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems can be interconnected via a system bus. Additional subsystems include a printer, keyboard, storage device(s), and monitor, which is coupled to display adapter. Peripherals and input/output (I/O) devices, which couple to I/O controller, can be connected to the computer system by any number of connections known in the art such as an input/output (I/O) port (e.g., USB, FireWire®. For example, an I/O port or external interface (e.g., Ethernet, Wi-Fi, etc.) can be used to connect computer system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

In some aspects, the present disclosure provides a non-transitory computer readable medium having stored thereon software instructions for treating social communication disorder (SCD) in a subject in need thereof that, when executed by a processor, cause the processor to sense, by a sensor in the electronic device, sound of social communication with the subject in an event. In some aspects, the present disclosure provides a non-transitory computer readable medium having stored thereon software instructions for treating social communication disorder (SCD) in a subject in need thereof that, when executed by a processor, cause the processor to provide the subject, by an electronic device, one or more first instructions for the subject to follow to improve social interaction, social cognition, and/or pragmatics, said one or more instructions based on one or more characteristics of the sound of the social communication.

Any of the software components or functions described in this application can be implemented as software code to be executed by a processor using any suitable computer language such as, for example, Java, C, C++, C #, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

Any of the methods described herein can be totally or partially performed with a computer system including one or more processors, which can be configured to perform the steps. Thus, embodiments can be directed to computer systems configured to perform the steps of any of the methods described herein, with different components performing a respective steps or a respective group of steps. Although presented as numbered steps, steps of methods herein can be performed at a same time or in a different order. Additionally, portions of these steps can be used with portions of other steps from other methods. Also, all or portions of a step can be optional. Additionally, any of the steps of any of the methods can be performed with modules, units, circuits, or other approaches for performing these steps.

### CERTAIN EMBODIMENTS

Embodiment 1. A method of treating social communication disorder (SCD) in a subject in need thereof, the method comprising: detecting, with an electronic device, sound of social communication with the subject in an event, wherein the electronic device comprises a sensor for sensing the sound of the social communication with the subject in the event and providing one or more first instructions for the subject to improve social interaction, social cognition, and/or pragmatics based on one or more characteristics of the sound of the social communication.

Embodiment 2. The method according to Embodiment 1, wherein the providing is performed within real-time or near-real-time of the event.

Embodiment 3. The method according to Embodiment 1 or 2, further comprising: sensing, using the sensor, adherence by the subject to the one or more first instructions, determining, based on the adherence, one or more second instructions for the subject to improve social interaction, social cognition, and/or pragmatics; and providing the one or more second instructions to the subject.

Embodiment 4. The method according to any one of Embodiments 1-3, wherein the sound is human voice.

Embodiment 5. The method according to any one of Embodiments 1-4, wherein the sound is voice of another subject in the social communication with the subject.

Embodiment 6. The method according to any one of Embodiments 1-5, further comprising analyzing the sound thereby determining the one or more characteristics of the sound.

Embodiment 7. The method according to any one of Embodiments 1-6, wherein the one or more characteristics are independently selected from the group consisting of vocabulary, syntax, phonology, voice wavering, voice frequency, rate of speech, word spacing, tone, voice pitch, voice amplitude, and coherence.

Embodiment 8. The method according to Embodiment 7, wherein the one or more characteristics comprises voice pitch.

Embodiment 9. The method according to Embodiment 7 or 8, further comprising analyzing the sound of the social communication to determine the one or more characteristics.

Embodiment 10. The method according to any one of Embodiments 1-6, wherein the one or more characteristics are independently selected from the group consisting of eye contact, eye movement, facial expressions, body language, and hand gestures.

Embodiment 11. The method according to any one of Embodiments 1-10, further comprising categorizing the one or more characteristics of the sound of the social communication as being associated with a standard response, a sarcastic response, a cynical response, an angry response, a sad response, a tense response, a pleasant response, an excited response, an accurate response, or an appropriate response.

Embodiment 12. The method according to Embodiment 11, wherein the accurate response or the appropriate response is determined by an expert in the field, a reviewer, a healthcare provider, or an artificial intelligence (AI).

Embodiment 13. The method according to Embodiment 11 or 12, wherein the accurate response or the appropriate response is determined based on information obtained from the user.

Embodiment 14. The method according to any one of Embodiments 1-13, wherein at least one of the sarcastic response, the cynical response, the angry response, the sad response, the tense response, the pleasant response, and the excited response is determined by an artificial intelligence (AI) when the vocabulary detected.

Embodiment 15. The method according to any one of Embodiments 1-13, wherein the sound of the social communication comprises at least one of speech from the subject, and speech from one or more other individuals involved in the social communication.

Embodiment 16. The method according to any one of Embodiments 1-15, further comprising determining the one or more first instructions for the subject to improve social interaction, social cognition, and/or pragmatics based on the categorizing.

Embodiment 17. The method according to any one of Embodiments 1-16, wherein the event is selected from the group consisting of an imaginary scenario, and a real-world event.

Embodiment 18. The method according to any one of Embodiments 1-17, further comprising scoring the social communication by the subject by comparing the one or more characteristics with a reference standard.

Embodiment 19. The method according to Embodiment 18, wherein the reference standard is determined using a pre-trained machine learning model.

Embodiment 20. The method according to Embodiment 19, wherein the pre-trained machine learning model is trained using a training data set comprising at least one of responses by healthy individuals and responses by individuals having the SCD.

Embodiment 21. The method according to Embodiment 20, further comprising providing a score to the subject.

Embodiment 22. The method according to any one of Embodiments 18-21, wherein the score is determined at least in part based on a self-evaluation by the subject following the event.

Embodiment 23. The method according to any one of Embodiments 18-22, wherein the one or more second instructions are determined based on the score.

Embodiment 24. The method according to any one of Embodiments 1-23, wherein the one or more first instructions and the one or more second instructions are independently selected from the group consisting of an alarm, a silent alarm or a vibration, an instruction to proceed, an instruction to stop, and instruction to avoid, and an instruction to maintain silence.

Embodiment 25. The method according to any one of Embodiments 1-24, wherein the electronic device is selected from the group consisting of a smartphone, an iPhone, an Android device, a smartwatch, a smart eyeglass, and a tablet.

Embodiment 26. A system for treating social communication disorder (SCD) in a subject in need thereof, comprising: an electronic device configured to: (i) detect sound of social communication with the subject in an event, wherein the electronic device comprises a sensor for sensing the sound of the social communication with the subject in the event and (ii) provide one or more first instructions for the subject to improve social interaction, social cognition, and/or pragmatics based on one or more characteristics of the sound of the social communication, a healthcare provider portal configured to provide one or more options to a healthcare provider to perform one or more tasks to prescribe treatment for social communication disorder (SCD) in the subject based on information received from the electronic device and an administrative portal configured to provide one or more options to an administrator of the system to perform one or more tasks to manage access to the system by the healthcare provider.

Embodiment 27. The system according to Embodiment 26, wherein the electronic device is configured to provide the one or more first instructions for the subject within real-time or near-real-time of the event.

Embodiment 28. The system according to Embodiment 26 or 27, wherein the electronic device is configured to: sense, using the sensor, adherence by the subject to the one or more first instructions, determine, based on the adherence, one or more second instructions for the subject to improve social interaction, social cognition, and/or pragmatics and provide the one or more second instructions to the subject.

Embodiment 29. The system according to any one of Embodiments 26-28, wherein the sound is human voice.

Embodiment 30. The system according to any one of Embodiments 26-29, wherein the sound is voice of another subject in the social communication with the subject.

Embodiment 31. The system according to any one of Embodiments 26-30, wherein the system is configured to execute a digital application for analyzing the sound to determine the one or more characteristics of the sound.

Embodiment 32. The system according to any one of Embodiments 26-31, wherein the one or more characteristics are independently selected from the group consisting of vocabulary, syntax, phonology, voice wavering, voice frequency, rate of speech, word spacing, tone, voice pitch, voice amplitude, and coherence.

Embodiment 33. The system according to Embodiment 32, wherein the one or more characteristics comprises voice pitch.

Embodiment 34. The system according to Embodiment 32 or 33, wherein the electronic device is configured to execute a digital application for analyzing the sound of the social communication to determine the one or more characteristics.

Embodiment 35. The system according to any one of Embodiments 26-34, wherein the one or more characteristics are independently selected from the group consisting of eye contact, eye movement, facial expressions, body language, and hand gestures.

Embodiment 36. The system according to any one of Embodiments 26-35, wherein the electronic device is configured to execute a digital application for categorizing the one or more characteristics of the sound of the social communication as being associated with a standard response, a sarcastic response, a cynical response, an angry response, a sad response, a tense response, a pleasant response, and excited response, an accurate response, or an appropriate response.

Embodiment 37. The system according to Embodiment 36, wherein the accurate response or the appropriate response is determined by an expert in the field, a reviewer, a healthcare provider, or an artificial intelligence (AI).

Embodiment 38. The system according to Embodiment 36, wherein the accurate response or the appropriate response is determined based on information obtained from the user.

Embodiment 39. The system according to Embodiment 38, wherein the digital application is configured to obtain the information from the user following event.

Embodiment 40. The system according to Embodiment 39, wherein the information comprises a numerical value or a qualitative assessment associated with the accuracy or the appropriateness of the subject's response to the event.

Embodiment 41. The system according to any one of Embodiments 26-40, wherein the sound of the social communication comprises at least one of speech from the subject, and speech from one or more other individuals involved in the social communication.

Embodiment 42. The system according to any one of Embodiments 26-41, wherein the electronic device is configured to execute a digital application for determining the one or more first instructions for the subject to improve social interaction, social cognition, and/or pragmatics based on the categorizing.

Embodiment 43. The system according to any one of Embodiments 26-42, wherein the event is selected from the group consisting of an imaginary scenario, and a real-world event.

Embodiment 44. The system according to any one of Embodiments 26-43, wherein the electronic device is configured to execute a digital application for scoring the social communication by the subject by comparing the one or more characteristics with a reference standard.

Embodiment 45. The system according to Embodiment 44, wherein the reference standard is determined using a pre-trained machine learning model.

Embodiment 46. The system according to Embodiment 45, wherein the pre-trained machine learning model is trained using a training data set comprising at least one of responses by healthy individuals and responses by individuals having the SCD.

Embodiment 47. The system according to Embodiment 46, wherein the electronic device is configured to provide the score to the subject.

Embodiment 48. The system according to any one of Embodiments 44-47, wherein the score is determined at least in part based on a self-evaluation by the subject following the event.

Embodiment 49. The system according to any one of Embodiments 44-48, wherein the one or more second instructions are determined based on the score.

Embodiment 50. The system according to any one of Embodiments 26-49, wherein the one or more first instructions and the one or more second instructions are independently selected from the group consisting of an alarm, a silent alarm or a vibration, an instruction to proceed, an instruction to stop, and instruction to avoid, and an instruction to maintain silence.

Embodiment 51. The system according to any one of Embodiments 26-50, wherein the electronic device is selected from the group consisting of a smartphone, a smartwatch, a smart eyeglass, and a tablet.

Embodiment 52. The system according to any one of Embodiments 26-51, wherein the one or more options provided to the healthcare provider are selected from the group consisting of adding or removing the subject, viewing or editing personal information for the subject, viewing adherence information for the subject, listening to a sound of the social communication with the subject in the event, viewing data associated with the one or more characteristics of the sound of the social communication, viewing a score of the social communication by the subject, altering a prescription for the subject, and communicating with the subject.

Embodiment 53. The system according to Embodiment 52, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

Embodiment 54. The system according to Embodiment 53, wherein the personal information comprises the prescription for the subject, and the prescription for the subject comprises one or more selected from the group consisting of a prescription identification number, a prescription type, a start date, a duration, and a completion date.

Embodiment 55. The system according to any one of Embodiments 26-54, wherein the one or more options provided to the administrator of the system are selected from the group consisting of adding or removing the healthcare provider, viewing or editing personal information for the healthcare provider, viewing or editing de-identified information of the subject, viewing adherence information for the subject, and communicating with the healthcare provider.

Embodiment 56. The system according to Embodiment 55, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

Embodiment 57. The system according to Embodiment 55, wherein the one or more options comprise the viewing or editing the de-identified information of the subject, and the de-identified information of the subject comprises one or more selected from the group consisting of an identification number for the subject, and the healthcare provider for the subject.

Embodiment 58. The system according to any one of Embodiments 26-57, wherein the electronic device comprises: a digital instruction generation unit configured to generate the one or more first instructions for the subject to improve social interaction, social cognition, and/or pragmatics based on one or more characteristics of the sound of the social communication, and provide the one or more first instructions to the subject and an outcome collection unit configured to collect adherence information comprising a sound of social communication from the subject after being provided the one or more first instructions.

Embodiment 59. The system according to Embodiment 58, wherein the digital instruction generation unit generates the one or more first instructions or the one or more second instructions based on inputs from the healthcare provider.

Embodiment 60. The system according to Embodiment 58, wherein the digital instruction generation unit generates the one or more first instructions or the one or more second instructions based on information received from the subject.

Embodiment 61. A computing system for treating social communication disorder (SCD) in a subject in need thereof, comprising: a sensor for detecting sound of social communication with the subject in an event and a digital instruction generation unit configured to provide, to the subject, one or more first instructions for the subject to follow to improve social interaction, social cognition, and/or pragmatics, said one or more instructions based on one or more characteristics of the sound of the social communication.

Embodiment 62. The computing system according to Embodiment 61, further comprising a transmitter configured to transmit adherence information to a server.

Embodiment 63. The computing system according to Embodiment 61 or 62, further comprising a receiver configured to receive, from the server, one or more second instructions based on the adherence information.

Embodiment 64. The computing system according to any one of Embodiments 61-63, wherein the digital instruction generation unit is configured to provide the one or more first instructions for the subject within real-time or near-real-time of the event.

Embodiment 65. The computing system according to any one of Embodiments 61-64, wherein the sensor is configured to sense adherence by the subject to the one or more first instructions.

Embodiment 66. The computing system according to Embodiment 65, wherein the digital instruction generation unit is configured to determine, based on the adherence, one or more second instructions for the subject to improve social interaction, social cognition, and/or pragmatics.

Embodiment 67. The computing system according to Embodiment 66, wherein the digital instruction generation unit is configured to provide the one or more second instructions to the subject.

Embodiment 68. The computing system according to any one of Embodiments 61-67, wherein the sound is human voice.

Embodiment 69. The computing system according to any one of Embodiments 61-68, wherein the sound is voice of another subject in the social communication with the subject.

Embodiment 70. The computing system according to any one of Embodiments 61-69, wherein the computing system is configured to execute a digital application for analyzing the sound to determine the one or more characteristics of the sound.

Embodiment 71. The computing system according to any one of Embodiments 61-67, wherein the one or more characteristics are independently selected from the group consisting of vocabulary, syntax, phonology, voice wavering, voice frequency, rate of speech, word spacing, tone, voice pitch, voice amplitude, and coherence.

Embodiment 72. The computing system according to Embodiment 71, wherein the one or more characteristics comprises voice pitch.

Embodiment 73. The computing system according to Embodiment 71 or 72, wherein the computing system is configured to execute a digital application for analyzing the sound of the social communication to determine the one or more characteristics.

Embodiment 74. The computing system according to any one of Embodiments 61-73, wherein the one or more characteristics are independently selected from the group consisting of eye contact, eye movement, facial expressions, body language, and hand gestures.

Embodiment 75. The computing system according to any one of Embodiments 61-74, wherein the computing system is configured to execute a digital application for categorizing the one or more characteristics of the sound of the social communication as being associated with a standard response, a sarcastic response, a cynical response, an angry response, a sad response, a tense response, a pleasant response, and excited response, an accurate response, or an appropriate response.

Embodiment 76. The computing system according to Embodiment 75, wherein the accurate response or the appropriate response is determined by an expert in the field, a reviewer, a healthcare provider, or an artificial intelligence (AI).

Embodiment 77. The computing system according to Embodiment 75, wherein the accurate response or the appropriate response is determined based on information obtained from the user.

Embodiment 78. The computing system according to Embodiment 77, wherein the digital application is configured to obtain the information from the user following event.

Embodiment 79. The computing system according to Embodiment 78, wherein the information comprises a numerical value or a qualitative assessment associated with the accuracy or the appropriateness of the subject's response to the event.

Embodiment 80. The computing system according to any one of Embodiments 61-79, wherein the sound of the social communication comprises at least one of speech from the subject, and speech from one or more other individuals involved in the social communication.

Embodiment 81. The computing system according to any one of Embodiments 61-80, wherein the computing system is configured to execute a digital application for determining the one or more first instructions for the subject to improve social interaction, social cognition, and/or pragmatics based on the categorizing.

Embodiment 82. The computing system according to any one of Embodiments 61-81, wherein the event is selected from the group consisting of an imaginary scenario, and a real-world event.

Embodiment 83. The computing system according to any one of Embodiments 61-82, wherein the computing system is configured to execute a digital application for scoring the social communication by the subject by comparing the one or more characteristics with a reference standard.

Embodiment 84. The computing system according to Embodiment 83, wherein the reference standard is determined using a pre-trained machine learning model.

Embodiment 85. The computing system according to Embodiment 84, wherein the pre-trained machine learning model is trained using a training data set comprising at least one of responses by healthy individuals and responses by individuals having the SCD.

Embodiment 86. The computing system according to Embodiment 85, wherein the digital instruction generation unit is configured to provide the score to the subject using a display or using a speaker.

Embodiment 87. The computing system according to any one of Embodiments 83-86, wherein the score is determined at least in part based on a self-evaluation by the subject following the event.

Embodiment 88. The computing system according to any one of Embodiments 83-87, wherein the one or more second instructions are determined based on the score.

Embodiment 89. The computing system according to any one of Embodiments 61-88, wherein the one or more first instructions and the one or more second instructions are independently selected from the group consisting of an alarm, a silent alarm or a vibration, an instruction to proceed, an instruction to stop, and instruction to avoid, and an instruction to maintain silence.

Embodiment 90. The computing system according to any one of Embodiments 61-89, wherein the computing system is selected from the group consisting of a smartphone, a smartwatch, a smart eyeglass, and a tablet.

Embodiment 91. A non-transitory computer readable medium having stored thereon software instructions for treating social communication disorder (SCD) in a subject in need thereof that, when executed by a processor, cause the processor to: sense, by a sensor in the electronic device, sound of social communication with the subject in an event and provide the subject, by an electronic device, one or more first instructions for the subject to follow to improve social interaction, social cognition, and/or pragmatics, said one or more instructions based on one or more characteristics of the sound of the social communication.

Embodiment 92. The non-transitory computer readable medium according to Embodiment 91, wherein the software instructions further cause the processor to transmit, by the electronic device, adherence information, based on the adherence, to a server.

Embodiment 93. The non-transitory computer readable medium according to Embodiment 91 or 92, wherein the software instructions further cause the processor to receive, from the server, one or more second instructions based on the adherence information.

Embodiment 94. The non-transitory computer readable medium according to any one of Embodiments 91-93, wherein the electronic device is configured to provide the one or more first instructions for the subject within real-time or near-real-time of the event.

Embodiment 95. The non-transitory computer readable medium according to any one of Embodiments 91-94, wherein the sensor is configured to sense adherence by the subject to the one or more first instructions.

Embodiment 96. The non-transitory computer readable medium according to Embodiment 95, wherein the electronic device is configured to determine, based on the adherence, one or more second instructions for the subject to improve social interaction, social cognition, and/or pragmatics.

Embodiment 97. The non-transitory computer readable medium according to Embodiment 96, wherein the electronic device is configured to provide the one or more second instructions to the subject.

Embodiment 98. The non-transitory computer readable medium according to any one of Embodiments 91-97, wherein the sound is human voice.

Embodiment 99. The non-transitory computer readable medium according to any one of Embodiments 91-98, wherein the sound is voice of another subject in the social communication with the subject.

Embodiment 100. The non-transitory computer readable medium according to any one of Embodiments 91-99, wherein the software instructions further cause the processor to analyze the sound thereby determining the one or more characteristics of the sound.

Embodiment 101. The non-transitory computer readable medium according to any one of Embodiments 91-100, wherein the one or more characteristics are independently selected from the group consisting of vocabulary, syntax, phonology, voice wavering, voice frequency, rate of speech, word spacing, tone, voice pitch, voice amplitude, and coherence.

Embodiment 102. The non-transitory computer readable medium according to Embodiment 101, wherein the one or more characteristics comprises voice pitch.

Embodiment 103. The non-transitory computer readable medium according to Embodiment 101 or 102, wherein the non-transitory computer readable medium is configured to execute a digital application for analyzing the sound of the social communication to determine the one or more characteristics.

Embodiment 104. The non-transitory computer readable medium according to any one of Embodiments 91-103, wherein the one or more characteristics are independently selected from the group consisting of eye contact, eye movement, facial expressions, body language, and hand gestures.

Embodiment 105. The non-transitory computer readable medium according to any one of Embodiments 91-104, wherein the software instructions further cause the processor to execute a digital application for categorizing the one or more characteristics of the sound of the social communication as being associated with a standard response, a sarcastic response, a cynical response, an angry response, a sad response, a tense response, a pleasant response, and excited response, an accurate response, or an appropriate response.

Embodiment 106. The non-transitory computer readable medium according to Embodiment 105, wherein the accurate response or the appropriate response is determined by an expert in the field, a reviewer, a healthcare provider, or an artificial intelligence (AI).

Embodiment 107. The non-transitory computer readable medium according to Embodiment 105, wherein the accurate response or the appropriate response is determined based on information obtained from the user.

Embodiment 108. The non-transitory computer readable medium according to Embodiment 107, wherein the digital application is configured to obtain the information from the user following event.

Embodiment 109. The non-transitory computer readable medium according to Embodiment 108, wherein the information comprises a numerical value or a qualitative assessment associated with the accuracy or the appropriateness of the subject's response to the event.

Embodiment 110. The non-transitory computer readable medium according to any one of Embodiments 91-109, wherein the sound of the social communication comprises at least one of speech from the subject, and speech from one or more other individuals involved in the social communication.

Embodiment 111. The non-transitory computer readable medium according to any one of Embodiments 91-110, wherein the software instructions further cause the processor to execute a digital application for determining the one or more first instructions for the subject to improve social interaction, social cognition, and/or pragmatics based on the categorizing.

Embodiment 112. The non-transitory computer readable medium according to any one of Embodiments 91-111, wherein the event is selected from the group consisting of an imaginary scenario, and a real-world event.

Embodiment 113. The non-transitory computer readable medium according to any one of Embodiments 91-112, wherein the software instructions further cause the processor to execute a digital application for scoring the social communication by the subject by comparing the one or more characteristics with a reference standard.

Embodiment 114. The non-transitory computer readable medium according to Embodiment 113, wherein the reference standard is determined using a pre-trained machine learning model.

Embodiment 115. The non-transitory computer readable medium according to Embodiment 114, wherein the pre-trained machine learning model is trained using a training data set comprising at least one of responses by healthy individuals and responses by individuals having the SCD.

Embodiment 116. The non-transitory computer readable medium according to Embodiment 115, wherein the software instructions further cause the processor to provide the score to the subject using a display or using a speaker of the electronic device.

Embodiment 117. The non-transitory computer readable medium according to any one of Embodiments 113-116, wherein the score is determined at least in part based on a self-evaluation by the subject following the event.

Embodiment 118. The non-transitory computer readable medium according to any one of Embodiments 113-117, wherein the one or more second instructions are determined based on the score.

Embodiment 119. The non-transitory computer readable medium according to any one of Embodiments 91-118, wherein the one or more first instructions and the one or more second instructions are independently selected from the group consisting of an alarm, a silent alarm or a vibration, an instruction to proceed, an instruction to stop, and instruction to avoid, and an instruction to maintain silence.

Embodiment 120. The non-transitory computer readable medium according to any one of Embodiments 91-119, wherein the non-transitory computer readable medium is contained within the electronic device, and wherein the electronic device is selected from the group consisting of a smartphone, a smartwatch, a smart eyeglass, and a tablet.

1. A method of treating social communication disorder (SCD) in a subject in need thereof, the method comprising:
detecting, with an electronic device, sound of social communication with the subject in an event, wherein the electronic device comprises a sensor for sensing the sound of the social communication with the subject in the event; and
providing one or more first instructions for the subject to improve social interaction, social cognition, and/or pragmatics based on one or more characteristics of the sound of the social communication.

2. The method of claim 1, wherein the providing is performed within real-time or near-real-time of the event.

3. The method of claim 1, further comprising:
sensing, using the sensor, adherence by the subject to the one or more first instructions;
determining, based on the adherence, one or more second instructions for the subject to improve social interaction, social cognition, and/or pragmatics; and
providing the one or more second instructions to the subject.

4. The method of claim 1, wherein the one or more characteristics are independently selected from the group consisting of vocabulary, syntax, phonology, voice wavering, voice frequency, rate of speech, word spacing, tone, voice pitch, voice amplitude, coherence, eye contact, eye movement, facial expressions, body language, and hand gestures.

5. The method of claim 1, further comprising categorizing the one or more characteristics of the sound of the social communication as being associated with a standard response, a sarcastic response, a cynical response, an angry response, a sad response, a tense response, a pleasant response, an excited response, an accurate response, or an appropriate response.

6. The method of claim 5, wherein the accurate response or the appropriate response is determined by an expert in the field, a reviewer, a healthcare provider, or an artificial intelligence (AI).

7. The method of claim 5, wherein the accurate response or the appropriate response is determined based on information obtained from the user.

8. The method of claim 5, wherein at least one of the sarcastic response, the cynical response, the angry response, the sad response, the tense response, the pleasant response, and the excited response is determined by an artificial intelligence (AI) when the vocabulary detected.

9. The method of claim 5, further comprising determining the one or more first instructions for the subject to improve social interaction, social cognition, and/or pragmatics based on the categorizing.

10. The method of claim 1, wherein the event is selected from the group consisting of an imaginary scenario, and a real-world event.

11. The method of claim 1, further comprising scoring the social communication by the subject by comparing the one or more characteristics with a reference standard.

12. The method of claim 11, wherein the reference standard is determined using a pre-trained machine learning model.

13. The method of claim 12, wherein the pre-trained machine learning model is trained using a training data set comprising at least one of responses by healthy individuals and responses by individuals having the SCD.

14. The method of claim 13, further comprising providing a score to the subject.

15. The method of claim 11, wherein the score is determined at least in part based on a self-evaluation by the subject following the event.

16. The method of claim 11, wherein the one or more second instructions are determined based on the score.

17. The method of claim 3, wherein the one or more first instructions and the one or more second instructions are independently selected from the group consisting of an alarm, a silent alarm or a vibration, an instruction to proceed, an instruction to stop, and instruction to avoid, and an instruction to maintain silence.

18. A system for treating social communication disorder (SCD) in a subject in need thereof, comprising:
the electronic device configured to perform the method of claim 1;
a healthcare provider portal configured to provide one or more options to a healthcare provider to perform one or more tasks to prescribe treatment for social communication disorder (SCD) in the subject based on information received from the electronic device; and
an administrative portal configured to provide one or more options to an administrator of the system to perform one or more tasks to manage access to the system by the healthcare provider.

19. A computing system for treating social communication disorder (SCD) in a subject in need thereof, comprising:
a sensor for detecting sound of social communication with the subject in an event; and
a digital instruction generation unit configured to provide, to the subject, one or more first instructions for the subject to follow to improve social interaction, social cognition, and/or pragmatics, said one or more instructions based on one or more characteristics of the sound of the social communication.

20. A non-transitory computer readable medium having stored thereon software instructions for treating social communication disorder (SCD) in a subject in need thereof that, when executed by a processor, cause the processor to perform the method of claim 1.
